(19) United States
(12) Patent Application Publication
(10) Pub. No.: US 2014/0276084 A1
(43) Pub. Date: Sep. 18, 2014

(54) INTRAVASCULAR ULTRASOUND DEVICES

(71) Applicant: VOLCANO CORPORATION, San Diego, CA (US)

(72) Inventor: Nathaniel J. Kemp, Concord, MA (US)

(73) Assignee: VOLCANO CORPORATION, San Diego, CA (US)

(21) Appl. No.: 14/205,789

(22) Filed: Mar. 12, 2014

Related U.S. Application Data
(60) Provisional application No. 61/781,515, filed on Mar. 14, 2013.

(51) Int. Cl. A61B 8/12; A61B 5/00; A61B 8/00

(52) U.S. Cl. CPC A61B 8/12; A61B 8/445; A61B 5/6851; A61B 8/4494
USPC 600/467

(57) ABSTRACT

Devices and methods of the invention provide for imaging an object within a body lumen. In certain aspects, a device for intraluminal imaging of the invention includes an elongate body defining a longitudinal axis, and a linear-array of imaging elements located on the body along the longitudinal axis.

### RELATED APPLICATION

This application claims the benefit of and priority to U.S. Provisional No. 61/781,515, filed Mar. 14, 2013, which is incorporated by reference in its entirety.

### FIELD OF THE INVENTION

The present invention generally relates to an imaging device for imaging the interior of a vessel.

### BACKGROUND

Intravascular ultrasound (IVUS) is an important interventional diagnostic procedure for imaging atherosclerosis and other vessel diseases and defects. The procedure involves the threading of an IVUS catheter over a guidewire into a blood vessel and the acquisition of images of the surrounding area using ultrasonic echoes. The three-dimensional images obtained via IVUS is often more informative than images derived from other imaging techniques, such as angiography, which provide only two dimensional images.

Conventional IVUS catheters come in various designs, including include phased-array IVUS or rotational IVUS catheters. In a rotational IVUS catheter, a single transducer having a piezoelectric crystal, which is located on a distal tip of an inner drive cable, is rapidly rotated while the transducer is intermittently excited with an electrical pulse. The excitation pulse causes the transducer to vibrate, sending out a series of transmit pulses. The transmit pulses are emitted at a frequency that allows time for receipt of echo signals. The sequence signals interspersed with receipt signals provides the ultrasound data required to reconstruct a complete cross-sectional image of a vessel. In order to image along the length of the vessel, the drive cable is pulled back during rotation of the catheter so that the linear array can obtain multiple cross-sectional frames of the vessel. The combined rotational/translational movement required to image along a length of the vessel risks inadvertent damage of the vessel walls.

Phased-array IVUS catheters include a transducer array that forms a circumferential ring around the distal end of the catheter device. With the ring-array configuration, the catheter does not to rotate to generate a cross-sectional image of a vessel. Signal processing is performed on the recorded acoustic signals to reconstruct an image (tomographic frame) whose orientation is perpendicular to the axis of the catheter body. Like rotational IVUS catheters, the phased-array catheter must be pulled back within the vessel lumen in order to provide multiple cross-sectional images across a length of the vessel.

### SUMMARY

The invention generally relates to systems and methods for obtaining images of an object whose orientation is parallel to an axis of an imaging device, without having to rotate or pull back the imaging device. Aspects of the invention are accomplished by providing a linear-phased array transducer along a longitudinal axis of a body of an imaging device. Using the linear-phased array transducer of the invention provides two-dimensional images of an object parallel to the imaging device without movement and provides three-dimensional images of a vessel with as little as single mechanical 360° rotation.

Devices of the invention include an elongate body defining a longitudinal axis, and a linear-array of imaging elements located on the elongate body along the longitudinal axis. In particular embodiments, the linear-array of imaging elements is a linear array of ultrasound transducers. The linear array of imaging elements may include any imaging element known in the art, such as ultrasound transducers, piezoelectric micro-machined ultrasound transducers, capacitive micro-machined ultrasound transducers, and photo-acoustic transducers.

According to certain aspects, methods of the invention involve introducing a device into a body lumen and imaging an object parallel to and extending a length of the body. The device involves a body defining a longitudinal axis, and a linear-array of imaging elements located on the body along the longitudinal axis. In one embodiment, the imaging is two-dimensional imaging. The two-dimensional imaging of the object may be achieved without translational movement and/or with rotational movement of the imaging element.

According to additional aspects, methods of the invention involve introducing a device into a body lumen, rotating the intraluminal device within the body lumen without translating the device along an axis of the body lumen, and generating a tomographic scan of the body lumen. The device involves a body defining a longitudinal axis, and a linear-array of imaging elements located on the body along the longitudinal axis. In one embodiment, the rotating step is a single 360° rotation of the device.

### BRIEF DESCRIPTION OF THE DRAWINGS

FIG. 1 depicts a prior art catheter.

FIG. 2 depicts a path of an imaging element of a prior art catheter during imaging.

FIG. 3 depicts A-scans received along the path depicted in FIG. 2.

FIG. 4 depicts A-scans obtained during imaging of a vessel wall with an imaging element following along the path depicted in FIG. 2.

FIG. 5 illustrates a set of A-scans A11, A12, . . . , A18 used to form a B scan according to certain embodiments of the invention.

FIG. 6 illustrates a B-scan generated the set of A-scan depicted in FIG. 5.

FIG. 7 illustrates a volumetric 3D image of a vessel wall created by a set of B-scans.

FIG. 8A depicts a rotating elongate member according to certain embodiments.

FIG. 8B depicts the rotating elongate member of FIG. 8A disposed within an outer catheter body.

FIG. 9 depicts the imaging planes of a linear-phased array configuration of the invention according to an embodiment.

FIG. 10 depicts the imaging planes of FIG. 9 with respect to imaging a vessel wall.

FIG. 11 depicts the movement of a catheter of the invention required to obtain a three-dimensional image.

FIG. 12 illustrates the imaging planes of a single-array imaging transducer during rotation.

FIG. 13 depicts a cross-sectional frame (B-scan) generated by each imaging plane of FIG. 12.

FIG. 14 exemplifies a catheter of the invention.

FIG. 15 is a system according to certain embodiments.

### DETAILED DESCRIPTION

The invention generally relates to systems and methods for obtaining images of an object whose orientation is parallel to an axis of an imaging device, without having to rotate or pull back the imaging device. Aspects of the invention are accomplished by providing a linear-phased array transducer along a longitudinal axis of a body of an imaging device. Using the linear-phased array transducer of the invention provides two-dimensional images of an object parallel to the imaging device without movement and provides three-dimensional images of a vessel with as little as single mechanical 360° rotation.

FIG. 1 depicts a prior art rotating catheter. The prior catheter includes a catheter sheath defining a lumen. A drive shaft is operably coupled to a housing that includes a single transducer array or imaging element. In order to image, the drive shaft is rotated during simultaneous translation motion, either forward or backward, along axis 117. This results in a motion for image capture described by FIG. 2. The single transducer sends signals to a luminal surface and receives reflected signals back from the luminal surface. Using acoustical energy, the single transducer sends acoustical energy in an array of A-scan lines as illustrated in FIG. 3 and detects reflected light. FIG. 4 shows the positioning of A-scans within a vessel obtained by a prior art rotating catheter. Each place where one of A-scans A11, A12, . . . , AN intersects a surface of a feature within vessel 101 (e.g., a vessel wall) where imaging signals (e.g. acoustic energy) is reflected and detected.

Data is collected from A-scans A11, A12, . . . , AN and stored in a tangible, non-transitory memory. Combined translational and rotational motion results in a three dimensional dataset of two dimensional image frames, where each frame provides a 360° slice of the vessel at different longitudinal locations. A set of A-scans captured in a helical pattern during a single 360° rotation of prior art around axis 117 collectively define a two-dimensional scan (B-Scan). FIG. 5 illustrates a set of A-scans A11, A12, . . . , A18 used to form a B scan according to certain embodiments of the invention. These A-scan lines are shown as would be seen looking down axis 117 (i.e., longitudinal distance between them is not shown). While eight A-scan lines are illustrated in FIG. 5, imaging applications may include between 100 and 1,000 A-scan lines to create a B scan (e.g., about 660). The data of all the A-scan lines together can be used to generate a two-dimensional image of a vessel cross-section. To create a final tomographic view of the vessel, the B-scan is scan converted to a Cartesian coordinate system, which is shown in FIG. 6. A collection of B-scans (created by rotation and pull-back of the single transducer) are used to create a three-dimensional image. FIG. 7 shows a three-dimensional graphic of a vessel using a collection of B-scans.

A disadvantage of the prior art catheter is that it requires both rotation and pull-back of the imaging element in order to generate a set of B-scan that can be used to generate a three-dimensional image. In addition, because only a single transducer is used to send and receive imaging signals, the effective resolution of prior art catheter is limited.

Devices of the invention overcome the limitations of the prior art by providing a rotational catheter including a plurality of imaging elements disposed along a length of an rotating elongate member. The plurality of imaging elements disposed along a length of a rotating elongate member are also described herein as a linear phased imaging array. FIGS. 8A and 8B depict a catheter of the invention. FIG. 8A depicts the rotating elongate member 103 of the catheter. The rotating elongate member includes a drive shaft 115 coupled to an imaging housing 120. The imaging housing 120 includes a plurality of imaging element 110. In certain embodiments, the plurality of imaging 110 is a transducer array. The plurality of imaging elements 110 disposed longitudinally along a length d of the elongate member 103. Preferably, the imaging elements 110 are arranged in a linear fashion. The linear longitudinal configuration is ideal because it eliminates the need to bend the plurality of imaging elements 110 around the circumference of the elongate member. This saves both manufacturing time and reduces design complexity of the imaging elements, for example array-based chip designs do not have to be rolled onto the array. The length d may be chosen depending on the application. In certain embodiments, the imaging array spans a length d of 0.5 mm, 1 mm, 5, mm, 1 cm, 1.5 cm, 2 cm, 2.5 cm, 3 cm, 3.5 cm, etc. In certain embodiments, the imaging array includes imaging elements placed side-by-side in a square/rectangular configuration. Alternatively, the imaging array may include a plurality of imaging elements in a single row. For example, the imaging array may include 5 imaging elements across and 10 imaging element disposed along the length. In certain embodiments, the plurality of imaging elements 110 is flexible so that the plurality of imaging elements 110 can maneuver through the curves in the vasculature. In certain embodiments, the imaging housing 120 may include two sets imaging elements, each set including a plurality of imaging elements disposed along a length of 2 of the elongate body 103.

FIG. 8B depicts the rotating elongate member 103 disposed within a lumen 135 of an outer catheter body 130. The outer catheter body 130 shields the vasculature from rotating motion of the elongate body 103. In addition, the outer catheter body 130 also protects the plurality of imaging elements from any disturbances in the vessel. As shown, the outer catheter body includes a rapid-exchange guidewire configured. Guidewire 125 is shown disposed within a distal end of the outer catheter body 130. The rotating elongate member 103 is configured to rotate within the outer catheter body 130.

Using the linear phased array configuration, the device of the invention is able to image an object in a plane parallel to the linear phased-array configuration without having to pull back rotating drive shaft (and thus the plurality of imaging elements). FIG. 9 depicts the imaging planes of a linear-phased array configuration of the invention according to one embodiment. The imaging plane shown in FIG. 9 is the imaging plane obtained when imaging while the rotating elongate body is stationary. The imaging elements are able to generate a set of imaging planes (B1, B2, . . . Bn). Each imaging plane represents the breadth signals sent and received by an imaging element, which form A-scans. FIG. 9 is only meant to exemplify the imaging planes of the linear phased array configuration. Depending on the number of imaging elements disposed in a linear configuration, one skilled in the art can appreciate that transducers in a phased array may generate multiple imaging planes, including overlapping imaging planes. The set of imaging planes (B1, B2, . . . Bn) may be used to generate an image parallel to the plurality of imaging elements, without the need to pull back or rotate the elongate member 103. Images of this type are especially helpful, for example, when viewing stent placement in a tight vessel where rotational movement is not desirable. FIG. 10 shows a portion of a vessel wall being imaged by the linear-phased imaging array. The data obtained from the imaging planes shown in FIG. 9 can be used to create a two-dimensional image of a length d of the vessel 1001 (corresponding to the length d of the linear transducer array in FIG. 9).

FIGS. 11-13 depict generation of a three-dimensional image of, e.g. a body lumen, using linear-phased imaging array of the invention. The rotational elongate member 103 is mechanically rotated. In certain embodiments, the rotational elongate member 103 is only rotated 360°. However, the rotational elongate member 103 may be rotated more than one time for multiple rotational data sets. FIG. 11 depicts the rotation of the rotational elongate member 103. Note that the linear-phased imaging array of the invention does not have to longitudinally translate within a body lumen to obtain a three-dimensional image (i.e. no pull-back or push-forward required). During rotation, each of the plurality of imaging elements collects cross-sectional data (or frame) of the lumen, as shown by the imaging plane of Bn being rotated 360°. Although only the imaging plane of Bn is shown rotated °360, each of B1, B2, . . . , Bn rotate 360° to form a slice of the vessel at those locations. As a result, a single rotation of the linear-phased imaging array (without translational motion) results in a three-dimensional data set of two dimensional image frames. Each frame represents a 360° slice of the vessel along the length of the vessel (B-scan), which is shown in FIG. 13. The frames (B-scans) can be combined to generate a three-dimensional volumetric model of a lumen, as shown in FIG. 7.

In addition, the linear-phased imaging array may be used to collect imaging data while being rotated and translated longitudinally. In this manner, the linear-phased imaging array is able to generate a large number of A-scans over a length of the vessel as compared to a single transducer device across the same length. As a result, the effective resolution of the array is beneficially increased.

The linear-phased imaging array of the invention includes a plurality of imaging elements disposed across a length d of the rotational elongate body. In certain embodiments, the imaging array includes imaging elements placed side-by-side in a square/rectangular configuration. Alternatively, the imaging array may include a plurality of imaging elements in a single row. In certain embodiments, the imaging elements of the array are transducers, such as ultrasound transducers, piezoelectric micromachined ultrasound transducers, capacitive micromachined ultrasound transducers, and photo-acoustic transducers. Each imaging elements of the array may include a signal transmitter and a signal collector (or image collector). The signal transducer and the signal collector may be the same or different. For example, a piezoelectric element that is used to transmit a signal may also be used to receive a signal. Ultrasound transducers produce ultrasound energy and receive echoes from which real time ultrasound images of a thin section of the blood vessel are produced. The transducers in the array may be constructed from piezoelectric components that produce sound energy at 20-50 MHz.

In yet another embodiment, the linear-phased imaging array is a plurality of optical acoustic imaging element. Optical-acoustic imaging elements include at least one acoustic-to-optical transducer. In certain embodiments, the acoustic-to-optical transducer is a Fiber Bragg Grating within an optical fiber. The linear-phased imaging array may include a single optical fiber with a plurality of Fiber Bragg Gratings along a length of the rotational elongate member. In addition, the linear phased imaging array may include two or more optical fibers aligned next to each other, and each with a plurality of Fiber Bragg Gratings. In some embodiments, the imaging elements may include an optical fiber with one or more Fiber Bragg Gratings (acoustic-to-optical transducer) and one or more other transducers. The at least one other transducer may be used to generate the acoustic energy for imaging. Acoustic generating transducers can be electric-to-acoustic transducers or optical-to-acoustic transducers. The imaging elements suitable for use in devices of the invention are described in more detail below.

Fiber Bragg Gratings for imaging provides a means for measuring the interference between two paths taken by an optical beam. A partially-reflecting Fiber Bragg Grating is used to split the incident beam of light into two parts, in which one part of the beam travels along a path that is kept constant (constant path) and another part travels a path for detecting a change (change path). The paths are then combined to detect any interferences in the beam. If the paths are identical, then the two paths combine to form the original beam. If the paths are different, then the two parts will add or subtract from each other and form an interference. The Fiber Bragg Grating elements are thus able to sense a change wavelength between the constant path and the change path based on received ultrasound or acoustic energy. The detected optical signal interferences can be used to generate an image using any conventional means.

Exemplary optical-acoustic imaging elements are disclosed in more detail in U.S. Pat. Nos. 6,659,957 and 7,527,594, 7,245.789, 7447,388, 7,660,492, 8,059,923 and in U.S. Patent Publication Nos. 2008/0119739, 2010/0087732 and 2012/0108943.

In another embodiment, the linear-phased imaging array of the invention is a capacitive micromachined ultrasound transducer array (CMUT). CMUT elements generally include at least a pair of electrodes separated by a uniform air or vacuum gap, with the upper electrode suspended on a flexible membrane. Impinging acoustic signals cause the membrane to deflect, resulting in capacitive changes between the electrodes, which produce electronic signals usable for ultrasonic imaging. Exemplary CMUT arrays are described in more detail in U.S. Pat. Nos. 8,309,428 and 6,328,696, and U.S. Publication No. 2007/0161896.

In another embodiment, the linear-phased imaging array of the invention is a piezoelectric micromachined ultrasound transducer array (PMUT). In PMUTs the sound-radiating element is a micromachined multi-layer membrane that is activated by a piezoactive layer (such as a PZT thin film). The PZT thin film is poled in the thickness direction. Application of an electric field across the thickness direction causes a strain in the film and induces membrane bending, thereby propagating a sound wave. reflected sound waves are received on the membrane, which causes a detectable charge displacement in the electrode PZT. PMUT arrays are described in more detail in U.S. Pat. No. 8,148,877, U.S. Publication No. 2003/0085635, and Akasheh, Firas, et al. “Development of piezoelectric micromachined ultrasonic transducers.” Sensors and Actuators A: Physical 111.2 (2004): 275-287.

FIG. 14 is a generalized depiction of a rotational imaging catheter 500 incorporating a linear-phased imaging array of the invention. Rotational imaging catheter 500 is typically around 150 cm in total length and can be used to image a variety of vasculature, such as coronary or carotid arteries and veins. When the rotational imaging catheter 500 is used, it is inserted into an artery along a guidewire (not shown) to the desired location. Typically a portion of catheter, including a distal tip 510, comprises a lumen (not shown) that mates with the guidewire, allowing the catheter to be deployed by pushing it along the guidewire to its destination.

An linear-phased imaging array 520 proximal to the distal tip 510, includes a plurality of transducers that image the tissue with ultrasound energy (e.g., 20-50 MHz range) and image collectors that collect the returned energy (echo) to create an intravascular image. The linear-phase imaging array 520 is configured to rotate and travel longitudinally within distal shaft 530 allowing the imaging assembly 520. The linear-phased array is able to generate three-dimensional images of the lumen with only rotational movement. The imaging assembly is rotated and manipulated longitudinally by a drive cable (not shown). In some embodiments of rotational imaging catheter 500, the distal shaft 530 can be over 15 cm long, and the imaging assembly 520 can rotate and travel most of this distance, providing thousands of images along the travel. Because of this extended length of travel, it is critical that the speed of acoustic waves through distal shaft 530 is properly matched, and that the interior surface of distal shaft 530 has a low coefficient of friction. In order to make locating the distal shaft 530 easier using angioscopy, distal shaft 530 optionally has radiopaque markers 537 spaced apart at 1 cm intervals.

Rotational imaging catheter 500 additionally includes proximal shaft 540 connecting the distal shaft 530 containing the linear phased imaging array 520 to the ex-corporal portions of the catheter. Proximal shaft 540 may be 100 cm long or longer. The proximal shaft 540 combines longitudinal stiffness with axial flexibility, thereby allowing a user to easily feed the catheter 500 along a guidewire and around tortuous curves and branching within the vasculature. The interior surface of the proximal shaft also has a low coefficient of friction, to reduce NURD, as discussed in greater detail above. The ex-corporal portion of the proximal shaft 540 may include shaft markers that indicate the maximum insertion lengths for the brachial or femoral arteries. The ex-corporal portion of catheter 500 also include a transition shaft 550 coupled to a coupling 560 that defines the external telescope section 565. The external telescope section 565 corresponds to the pullback travel, which is on the order of 150 mm. The telescoping feature may be used to position the linear phased imaging array 520 into position for imaging. The end of the telescope section is defined by the connector 570 which allows the catheter 500 to be interfaced to an interface module which includes electrical connections to supply the power to the transducer and to receive images from the image collector. The connector 570 also includes mechanical connections to rotate the imaging assembly 520. When used clinically, pullback of the imaging assembly is also automated with a calibrated pullback device (not shown) which operates between coupling 2560 and connector 570.

In some embodiments, a device of the invention includes a linear-phase imaging array and obtains a three-dimensional data set through the operation of IVUS, or other imaging hardware. In some embodiments, a device of the invention is a computer device such as a laptop, desktop, or tablet computer, and obtains a three-dimensional data set by retrieving it from a tangible storage medium, such as a disk drive on a server using a network or as an email attachment.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

In some embodiments, a user interacts with a visual interface to view images from the imaging system. Input from a user (e.g., parameters or a selection) are received by a processor in an electronic device. The selection can be rendered into a visible display. An exemplary system including an electronic device is illustrated in FIG. 15. As shown in FIG. 15, an imaging engine 859 of the imaging assembly communicates with host workstation 433 as well as optionally server 413 over network 409. The data acquisition element 855 (DAQ) of the imaging engine receives imaging data from one or more imaging element. In some embodiments, an operator uses computer 449 or terminal 467 to control system 400 or to receive images. An image may be displayed using an I/O 454, 437, or 471, which may include a monitor. Any I/O may include a keyboard, mouse or touchscreen to communicate with any of processor 421, 459, 441, or 475, for example, to cause data to be stored in any tangible, nontransitory memory 463, 445, 479, or 429. Server 413 generally includes an interface module 425 to effectuate communication over network 409 or write data to data file 417.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 413), a middleware component (e.g., an application server), or a front-end component (e.g., a client computer 449 having a graphical user interface 454 or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a portion of file 417 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein

What is claimed is:

1. A device for intraluminal imaging, the device comprising
an elongate body defining a longitudinal axis and configured for insertion into vasculature of a patient; and
a linear-array of imaging elements located on the elongate body along the longitudinal axis.

2. The device of claim 1, wherein the imaging elements comprise ultrasound transducers.

3. The device of claim 1, wherein the device is a catheter.

4. The device of claim 1, wherein the device is a guidewire.

5. The device of claim 1, wherein the imaging elements, when the device is inserted into a vessel, is able to image an object parallel to and extending a length of the elongate body.

6. The method of claim 5, wherein two-dimensional imaging of the object does not require translational movement of the linear array of imaging elements.

7. The method of claim 5, wherein two-dimensional imaging of the object does not require rotational movement of the linear array of imaging elements.

8. A method for intraluminal imaging, the method comprising
inserting a device into a body lumen, wherein the device comprises
an elongate body defining a longitudinal axis; and
a linear-array of imaging elements located on the elongate body along the longitudinal axis; and
imaging an object parallel to and extending a length of the elongate body.

9. The method of claim 8, wherein two-dimensional imaging of the object does not require translational movement of the linear array of imaging elements.

10. The method of claim 8, wherein two-dimensional imaging of the object does not require rotational movement of the linear array of imaging elements.

11. The method of claim 8, wherein the imaging elements comprise ultrasound transducers.

12. The method of claim 8, wherein the device is a catheter.

13. The method of claim 8, wherein the device is a guidewire.

14. A method for intraluminal imaging, the method comprising
inserting a device into a body lumen, wherein the device comprises
an elongate body defining a longitudinal axis; and
a linear-array of imaging elements located on the elongate body along the longitudinal axis;
rotating the intraluminal device within the body lumen without translating the device along an axis of the body lumen;
generating a tomographic scan of the body lumen.

15. The method of claim 14, wherein the rotating step is a single 360° rotation of the device.

16. The method of claim 14, wherein the imaging elements comprise ultrasound transducers.
